(19) United States
(12) Patent Application Publication
(10) Pub. No.: US 2013/0108980 A1
(43) Pub. Date: May 2, 2013

(54) INTRA-ORAL IMAGER ISOLATING DEVICE

(71) Applicant: Chris Leinweber, Calgary (CA)

(72) Inventor: Chris Leinweber, Calgary (CA)

(21) Appl. No.: 13/809,888

(22) PCT Filed: Jun. 27, 2011
(86) PCT No.: PCT/IB11/52822

§ 371 Date: Jan. 12, 2013

Related U.S. Application Data
(60) Provisional application No. 61/399,616, filed on Jul. 15, 2010.

(51) Int. Cl. A61B 1/24

(52) U.S. Cl. CPC A61B 1/24
USPC 433/29

(57) ABSTRACT

An isolating device for an intra-oral imager includes a body which is configured to receive the intra-oral imaging device such that the isolating device is positioned about the imaging optics of the intra-oral imaging device. The body includes a pair of lateral isolating wings that provide a tooth receiving space into which is positionable a tooth surface to be imaged by the intra-oral imaging device with the isolating wings extending along opposite sides of the dental arch. The isolating wings isolate the tooth surface disposed within the tooth receiving space from adjacent oral anatomy and further provide stabilization to the intra-oral imaging device during imaging of the tooth surface.

### FIELD OF THE INVENTION

The present invention relates generally to devices for intra-oral imaging, and more particularly, relating to an isolating device for intra-oral imagers that prevents contact between a patient's tongue and cheek with a tooth surface during imaging of the tooth surface and which aids in stabilizing an intra-oral imager during image capture.

### BACKGROUND OF THE INVENTION

Intra-oral imaging, and particularly 3D imaging of tooth surfaces is desirable for many purposes including the production of prosthodontics or dental prosthetics such as crowns, bridges, veneers, and dentures, implant restorations, etc. Digital dental impression (3D imaging of tooth surfaces) eliminates the need for physical dental impressions and further can be used in computer assisted manufacturing (CAM) of prosthodontics resulting in a higher quality product with a more accurate fit.

A digital dental impression is made utilizing an intra-oral imaging device that is inserted into the patient's oral cavity and is operated to take a series of images of a tooth surface which are then constructed into a 3D image of the tooth surface. To ensure accurate imaging, the intra-oral imaging device must remain stationary during imaging, the tooth surface being imaged needs to remain dry and needs to be isolated against contact from adjacent oral anatomy, such as the cheek, tongue and/or lips. In certain imaging processes, it is additionally desirable to limit reflection of ambient light from the tooth surface being imaged.

### SUMMARY OF THE INVENTION

Embodiments of the present invention provide an isolating device for an intra-oral imaging device including a tooth receiving space which isolates a tooth surface disposed within the tooth receiving space from contact with adjacent oral anatomy.

Embodiments of the present invention provide an isolating device for an intra-oral imaging device including lateral isolating wings which provide a tooth receiving space and provide stability to the intra-oral imaging device to which the isolating device is secured.

Embodiments of the present invention provide an isolating device for an intra-oral imaging device that reduces ambient light reflection from tooth surfaces.

Embodiments of the present invention provide an isolating device for an intra-oral imaging device that provides alignment to the intra-oral imaging device during positioning within in an oral cavity.

Embodiments of the present invention also provide an isolating device for an intra-oral imaging that provides cheek and tongue retraction without the use of dams and clamps.

To achieve these and other advantages, in general, in one aspect, an isolating device for an intra-oral imaging device having a tip end and further having imaging optics that are disposed at the tip end that are inserted into an oral cavity for imaging tooth surfaces therein is provided. The isolating device includes a body configured to receive the tip end of an intra-oral imaging device and first and second isolating wings extending from the body and defining a tooth receiving space therebetween.

In general, in another aspect, the first and the second isolation wings longitudinally extend along opposite sides of the body and generally in a direction that is parallel to a longitudinal length of the tip end of the intra-oral imaging device when received by the body.

In general, in another aspect, the body includes an intra-oral device tip end receiving passage which the tip end of the intra-oral device extends when the tip end is received by the body.

In general, in another aspect, the isolating device may further include a distal fulcrum extending from the body.

In general, in another aspect, the isolating device may further include a mesial fulcrum extending from the body.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The following drawings illustrate by way of example and are included to provide further understanding of the invention for the purpose of illustrative discussion of the embodiments of the invention. No attempt is made to show structural details of the embodiments in more detail than is necessary for a fundamental understanding of the invention, the description taken with the drawings making apparent to those skilled in the art how the several forms of the invention may be embodied in practice. Identical reference numerals do not necessarily indicate an identical structure. Rather, the same reference numeral may be used to indicate a similar feature of a feature with similar functionality. In the drawings:

FIG. 1 is a perspective view of an embodiment of an isolating device in accordance with the principles of the invention with a representatively illustrated intra-oral imaging device;

FIG. 2 is a cross sectional view of the isolating device and intra-oral imaging device taken along line 2-2 in FIG. 1;

FIG. 3 is a cross sectional view of the isolating device and intra-oral imaging device taken along line 3-3 in FIG. 2;

FIG. 4 is a perspective view of an isolating device in accordance with an embodiment of the invention;

FIG. 5 is a top view of an isolating device in accordance with an embodiment of the invention;

FIG. 6 is a back view of an isolating device in accordance with an embodiment of the invention;

FIG. 7 is a front view of an isolating device in accordance with an embodiment of the invention;

FIG. 8 is perspective view of an isolating device in accordance with an alternative embodiment of the invention;

FIG. 9 is an alternative perspective view of the isolating device of FIG. 8;

FIG. 10 is an alternative perspective view of the isolating device of FIG. 8; and

FIG. 11 is a rear view of the isolating device of FIG. 8.

### DETAILED DESCRIPTION OF THE INVENTION

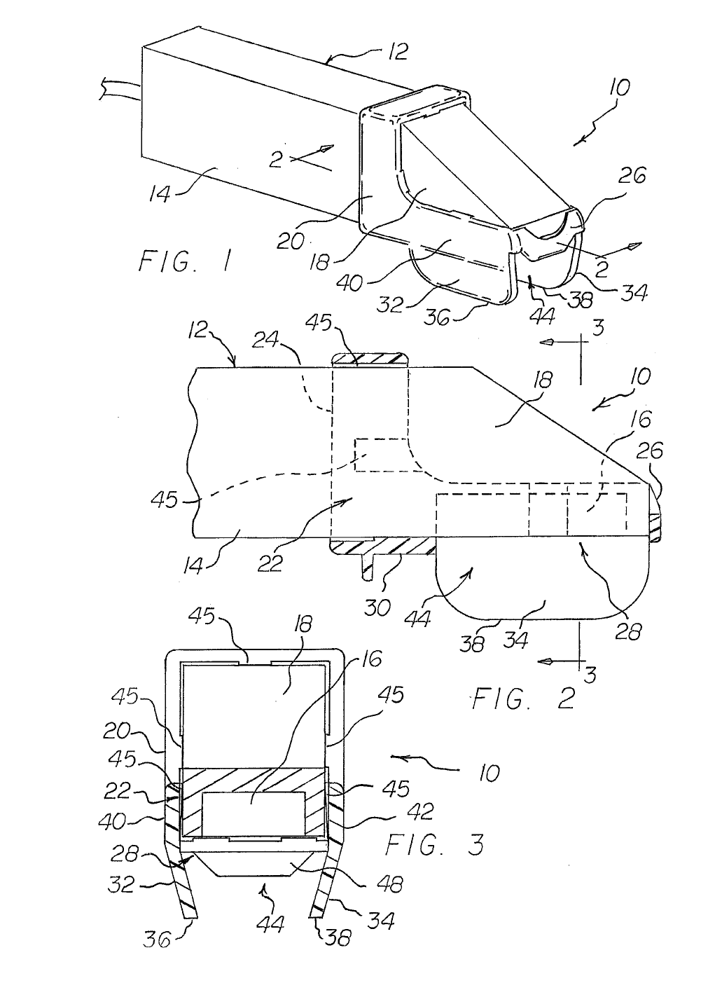

Referring to FIGS. 1 and 2, there is illustrated an intra-oral isolating device embodying the principles of the invention and generally designated by reference number 10. As illustrated, isolating device 10 is shown with a representatively illustrated intra-oral imaging device 12 of a known configuration and which forms no part of the invention. The intra-oral imaging device 12 is an imaging device that is conventionally utilized in imaging tooth surfaces within the oral cavity of an individual to render a 3-dimensional image of tooth surfaces, which may also be referred to as a digital dental impression. The digital dental impression is used to create dental prosthetics such as crowns, bridges, veneers, and dentures, implant restorations, etc. Conventionally, the intra-oral imaging device 12 includes an elongated wand or shaft 14 that is configured to be grasped by an operator during use, e.g. during positioning and imaging of tooth surfaces within an oral cavity. The intra-oral imaging device 12 further includes image optics 16 disposed at a distal tip end 18 of the shaft 14. The image optics 16 and the tip end 18 are configured such that imaging takes places across a surface of the shaft 14, such as a bottom surface of the shaft as illustrated herein. The image optics 16 may vary between intra-oral imaging device types.

Isolating device 10 is generally of a sleeve construction which is configured to receive the tip end 18 of the intra-oral imaging device 12. The isolating device 10 is configured to provide stabilization to the intra-oral imaging device 12 when inserted into an oral cavity during imaging and to provide tongue and cheek isolation from the tooth surfaces during imaging.

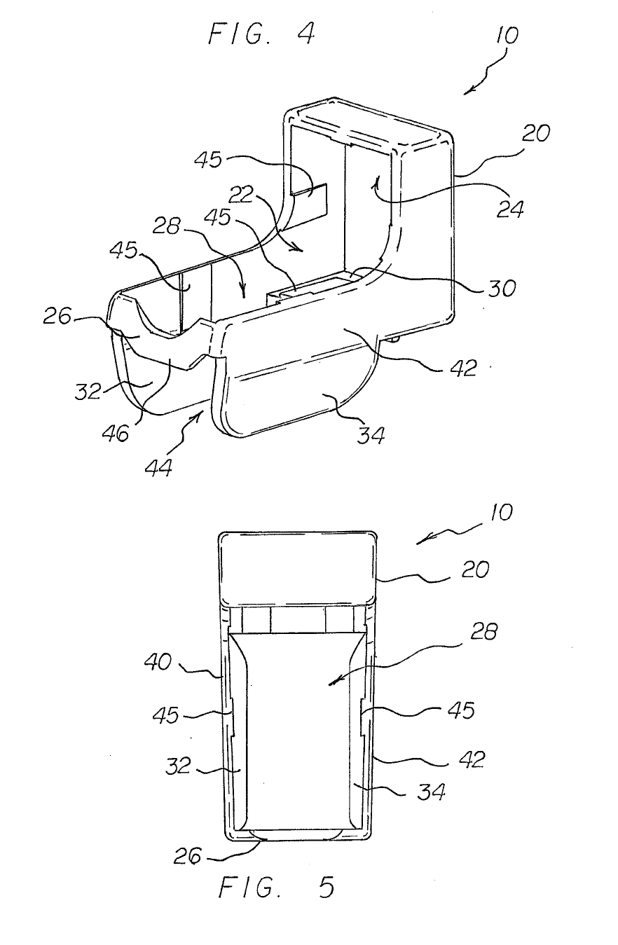

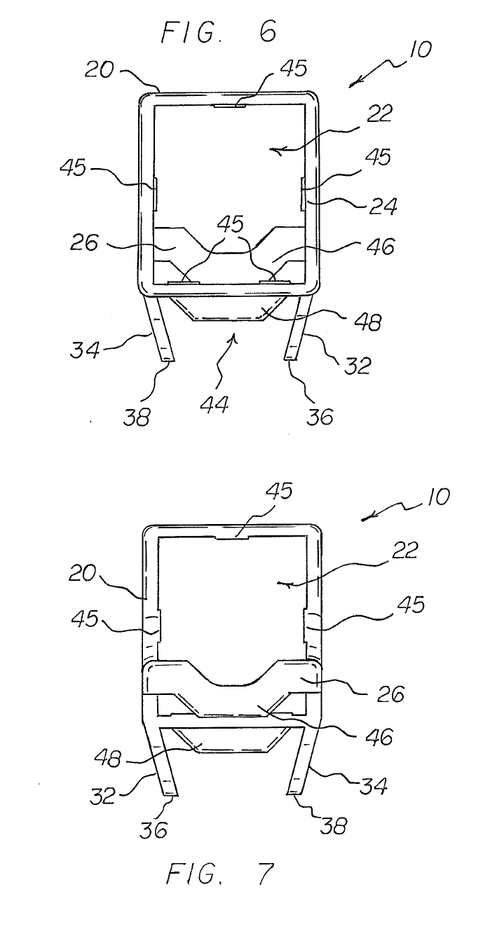

With reference to FIGS. 1 through 7, isolating device 10 is comprised of a body 20 of a unitary construction that is configured to receive at least the tip end 18 of the intra-oral imaging device 12. Body 20 may be configured to frictionally receive the tip end 18 of the intra-oral imaging device 12, thereby frictionally retaining the intra-oral imaging device and the body together in an assembly for use and insertion into an oral cavity of a patient.

Body 12 includes a tip end receiving passage 22 that is open at end 24 and at least partially closed at an opposite end 26. The tip end 18 of the intra-oral imaging device 12 extends the tip end receiving passage 22 through open end 24 and abuts closed end 26. As further illustrated, body 12 encircles the tip end 18 of the intra-oral imaging device 12 along the at least a partial length of the tip end receiving passage 22, for example at the proximal or mesial end of the body 12.

Body 12 further includes an aperture 28 which may extend through a wall 30 of the tip end receiving passage 22. Aperture 28 is configured such that the image optics 16 of the intra-oral imaging device 12 are registered with the aperture when the tip end 18 is fully received by the tip end receiving passage 22. In this manner, the image optics 16 are unobstructed by the body 12 for imaging tooth surfaces.

Body 12 further includes first and second tongue and cheek isolating wings 32 and 34. Isolating wings 32 and 34 extend from body 12 in generally the same direction and terminate at ends 36 and 38, respectively. Isolating wings 32 and 34 extend along opposing longitudinal sides 40 and 42, respectively, of body 12 and define a tooth receiving space 44 therebetween. Depending upon orientation in use, isolating wings 32 and 34 isolate the buccal surfaces of the tooth from the cheeks and isolate the lingual surface of the tooth from the tongue when the tooth surfaces are disposed within the tooth receiving space 44. Additionally, isolating wings 32 and 34 may be used to separate the upper and lower lips to provide unobstructed anterior imaging. Preferably, the isolating wings 32 and 34 extend at least along the entire length of aperture 28 to provide complete tooth surface isolation and prevent contact with the imaging optics 16 by the tongue and/or cheek.

In an embodiment, isolating wings 32 and 34 may also serve to stabilize the intra-oral imaging device 12 during use. In this embodiment, the terminal ends 36 and 38 of the isolating wings 32 and 34, respectively may be inwardly curved forming a tooth receiving space 44 that tapers from wide to narrow in a direction towards the terminal ends to provide stabilization to the intra-oral imaging device 12 by resting terminal ends 36 and 38 on opposite sides of the intra-oral arch along the tooth surfaces being imaged.

Isolating wings 32 and 34 also limit ambient light reflection from tooth surfaces during image capture by preventing ambient light from entering the imaging frame from the buccal and lingual sides. Limiting ambient light reflection is desirable in certain imaging processes, for example, during inferred imaging of the tooth surfaces.

Additionally, isolating wings 32 and 34 may be used to separate the upper and lower lips to provide unobstructed anterior imaging.

Isolating device 10 further includes a distal fulcrum tab 46 and optionally a mesial fulcrum tab 48 which serve as supports to the intra-oral imaging device 12 by resting or positioning the fulcrum tabs on tooth surfaces that are disposed adjacent to the tooth surfaces being imaged. In an embodiment, the distal fulcrum tab 46 extends from a distal end of the body 12 and generally in the direction of isolating wings 32 and 34. Similarly, in an embodiment, the mesial tab 48 extends from a proximal or mesial end of the body 12 and generally in the direction of the isolating wings 32 and 34. In embodiments, the fulcrum tabs 46 and 48 terminate at an inwardly disposed location relative to the terminal ends 36 and 38 of the isolating wings 32 and 34, respectively.

In embodiments, the isolating device 10 may be made of a rigid or flexible material, and preferably from a flexible plastic or polymer material. In embodiments, the isolating device 10 may be made to be reused and withstand sanitization for example by an autoclave. In embodiments, the isolating device 10 may be made to be disposed of after use.

In embodiments, the isolating device 10 may include a plurality of wedge-shaped surfaces 45 arranged along receiving passage 22 which act to frictionally engage the exterior surface of the imaging device 12 during reception by the receiving passage, thereby removably joining body 12 and the imaging device in a frictional engagement.

Operation of isolating device 10 is readily apparent from the above description, and thus a detailed description of the operation of the isolating device is not required herein.

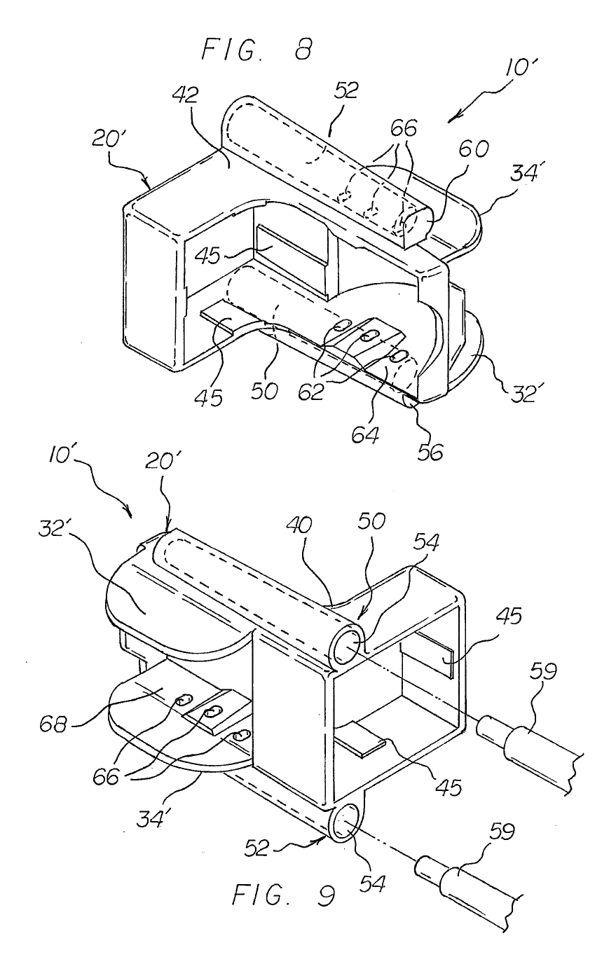

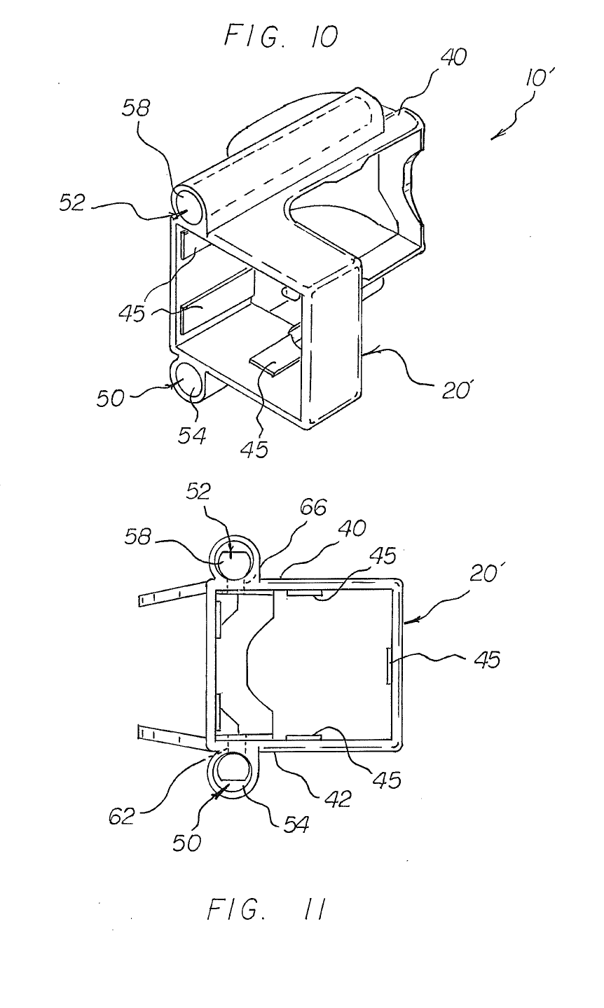

With reference now to FIGS. 8 through 11, there is illustrated an alternative embodiment of an isolating device embodying the principles of the invention and generally designated by reference number 10′. In reference to FIGS. 8 through 11, the same reference numbers refer to similar elements of isolating device 10. Isolating device 10′ is configured for connection to a conventional suction/air supply device (not illustrated) for the clearing of a target site through either suction and/or air as desired by the operator of the isolating device prior to or post imaging of the target site.

As illustrated, isolating device 10′ is similar to isolating device 10, but further includes suction/air passages or fluid passages 50 and 52 integrated into the body 20′. Passages 50 and 52 extend along opposite sides 40 and 42 of body 20′. Passage 50 is open at end 54 and closed at end 56, thereof. Likewise, passage 52 is open at end 58 and closed at end 60, thereof. One or more openings 62 extend through an interior facing surface 64 of isolating wing 32′ and into passage 50. Similarly, one or more openings 66 extend through an interior facing surface 68 of isolating wing 34′ and into passage 52.

Passages 50 and 52, for example at open ends 54 and 58, respectively, are configured for connection to one or more suction/air supply lines 59 to connect passages 50 and 52 to a conventional suction/air supply device (not shown). Accordingly, a supply of suction or a supply of air may be provided at openings 62 and 66 for either suctioning the target area or clearing the target area by an air stream through operation of the suction/air supply device in a conventional manner.

A number of embodiments of the present invention have been described. Nevertheless, it will be understood that various modifications may be made without departing from the spirit and scope of the invention. Accordingly, other embodiments are within the scope of the following claims.

What is claimed is:

1. An intra-oral imager isolating device for an intra-oral imager having a tip end and further having imaging optics that are disposed at the tip end that are inserted into an oral cavity for imaging tooth surfaces, the device comprising:
a body configured to receive the tip end of the intra-oral imager; and
first and second isolating wings extending from opposite sides said body and define a tooth receiving space therebetween with which the imaging optics of the intra-oral imager is registered when the tip end of the intra-oral imager is received by said body.

2. The isolating device of claim 1, wherein said body includes an intra-oral imager tip end receiving passage which the tip end of the intra-oral imager extends when the tip end is received by said body.

3. The isolating device of claim 1, wherein said first and said second isolation wings longitudinally extend along opposite sides of said body and generally in a direction that is parallel to a longitudinal length of the tip end of the intra-oral imaging device when received by said body.

4. The isolating device of claim 1, wherein said first and second isolating wings are inwardly curved.

5. The isolating device of claim 1, further comprising:
a distal fulcrum extending from said body.

6. The isolating device of claim 5, further comprising:
a mesial fulcrum extending from said body.

7. The isolating device of claim 1, wherein said body further includes an aperture with which the imaging optics of the intra-oral imager is registered when the tip end is received by body to image tooth surfaces that are positioned within said tooth space.

8. The isolating device of claim 7, wherein said first and said second isolation wings extend the entire longitudinal length of said aperture.

9. The isolating device of claim 7, wherein said first and said second isolation wings longitudinally extend along opposite sides of said body and generally in a direction that is parallel to a longitudinal length of the tip end of the intra-oral imager when the intra-oral imager is received by said body.

10. The isolating device of claim 7, wherein said first and second isolating wings are inwardly curved.

11. The isolating device of claim 7, further comprising:
a distal fulcrum extending from said body.

12. The isolating device of claim 11, further comprising:
a mesial fulcrum extending from said body.

13. The isolating device of claim 1, further comprising:
one or more fluid passages defined by said body; and
one or more fluid discharge openings into said one or more fluid passages.

14. The isolating device of claim 13, wherein said body further includes an aperture with which the imaging optics of the intra-oral imager is registered when the tip end is received by body to image tooth surfaces that are positioned within said tooth space.

15. The isolating device of claim 14, wherein said first and said second isolation wings extend the entire longitudinal length of said aperture.

16. The isolating device of claim 13, wherein said first and said second isolation wings longitudinally extend along opposite sides of said body and generally in a direction that is parallel to a longitudinal length of the tip end of the intra-oral imager when the intra-oral imager is received by said body.

17. The isolating device of claim 13, wherein said first and second isolating wings are inwardly curved.

18. The isolating device of claim 13, further comprising:
a distal fulcrum extending from said body.

19. The isolating device of claim 18, further comprising:
a mesial fulcrum extending from said body.

20. The isolating device of claim 13, wherein said one or more openings extend through an interior surface of one or both of said first and second isolating wings.

21. The isolating device of claim 20, wherein said body further includes an aperture with which the imaging optics of the intra-oral imager is registered when the tip end is received by body to image tooth surfaces that are positioned within said tooth space.

22. The isolating device of claim 21, wherein said first and said second isolation wings extend the entire longitudinal length of said aperture.

23. The isolating device of claim 20, wherein said first and said second isolation wings longitudinally extend along opposite sides of said body and generally in a direction that is parallel to a longitudinal length of the tip end of the intra-oral imager when the intra-oral imager is received by said body.

24. The isolating device of claim 20, wherein said first and second isolating wings are inwardly curved.

25. The isolating device of claim 20, further comprising:
a distal fulcrum extending from said body.

26. The isolating device of claim 20, further comprising:
a mesial fulcrum extending from said body.
